(19) United States
(12) Patent Application Publication
(10) Pub. No.: US 2005/0096557 A1
(43) Pub. Date: May 5, 2005

(54) Noninvasive cardiovascular monitoring methods and devices

(76) Inventors: Frederick Vosburgh, Durham, NC (US); Walter C. Hernandez, Potomac, MD (US); Mathieu Kemp, Durham, NC (US)

Correspondence Address: MYERS BIGEL SIBLEY & SAJOVEC, PO BOX 37428, RALEIGH, NC, 27627, US

(21) Appl. No.: 10/753,050

(22) Filed: Jan. 7, 2004

Related U.S. Application Data
(60) Provisional application No. 60/438,743, filed on Jan. 8, 2003.
(60) Provisional application No. 60/514,851, filed on Oct. 27, 2003.

(51) Int. Cl. A61B 5/04

(52) U.S. Cl. 600/509

(57) ABSTRACT

Monitoring the physiologic status of a human or animal subject includes detecting a blood vessel signal with a sensor. A physiologic time interval can be determined, and information related to the physiologic status of the subject can be analyzed and communicated.

### RELATED APPLICATIONS

This application claims priority to U.S. Provisional Application Ser. No. 60/438,743, filed Jan. 8, 2003 and U.S. Provisional Application Ser. No. 60/514,851, filed Oct. 27, 2003, the disclosures of which are hereby incorporated by reference in their entireties.

### FIELD OF THE INVENTION

This invention relates generally to methods and devices for cardiovascular monitoring.

### BACKGROUND OF THE INVENTION

Trauma is the leading cause of premature death in the United States. Research shows that triage decisions in the first 30 minutes following injury may strongly influence survival. Triage of multiple trauma victims, such as in the wake of a highway catastrophe or a terrorist attack, presents a particular problem for a first responder having limited trauma management resources. Automated technology to aid triage can result in substantial social and economic benefit.

Systolic blood pressure below 90 mm Hg may indicate shock in trauma that, unresolved, can lead quickly to death. While invasive catheters are used for physiologic monitoring in the operating room or intensive care unit, blood pressure cuffs are the predominantly used devices in other settings. The cuff interrupts arterial flow while heart sounds are detected with a stethoscope, or arterial pulsations are detected by an electromechanical transducer.

Devices using pressure cuffs provide readings infrequently relative to how quickly a patient can decompensate. In addition, listening for heart sounds requires skill and attention, and can be particularly difficult in unsettled situations in which multiple victims require attention. Automated cuff devices and other pulsation detectors are widely known to be prone to motion artifacts. Cuffs and optical sensors placed on the finger are of little value for detecting hypotensive shock given the autonomic shutdown of peripheral capillary circulation that may occur in trauma victims.

Blood pressure can be measured using pulse wave velocity, as disclosed in U.S. Pat. No. 5,865,755. Methods for detecting pulse wave velocity typically require two detection points such as two spaced apart blood vessel detection points or an electrocardiogram (EKG) combined with a blood vessel detection point. However, these methods have failed to gain widespread acceptance for at least two reasons. First, devices typically require calibration for each use to provide an absolute blood pressure measurement. Second, considerable skill and attention may be required to detect arterial signals continuously without interruption when using existing sensors.

Various devices have been proposed for detecting pulse by electromechanical, optical, or ultrasonic techniques. U.S. Pat. No. 4,830,014 discloses a single optical sensor having an adhesive strip to prevent dislodging. U.S. Pat. No. 6,334,850 discloses manual moving of a sensor to adjust its position with respect to a vessel to find a signal. U.S. Pat. No. 6,447,456, employs one sensor over the radial artery and a second sensor over the ulnar artery to reduce the frequency of times when no signal can be detected. U.S. Pat. No. 6,371,920 discloses a rigid array having an expanded field of view.

Placement and motion artifacts remain problematic for automatic cuff devices, tonometers, and ultrasonic sensors alike, requiring a skilled health care professional for proper use. Furthermore, taking manual readings can be time consuming, which is undesirable in an emergency situation.

### SUMMARY OF THE INVENTION

The present invention is directed to devices and methods to detect physiologic changes that can address some of the issues noted above. In certain embodiments, a method and noninvasive device to determine pulse wave intervals is provided. Embodiments of the invention may be applicable to cardiovascular signals in determining pulse wave parameters. Devices according to embodiments of the invention may be motion and placement tolerant to facilitate the acquisition of such signals. In some embodiments, methods for monitoring the physiologic status of a human or animal subject include detecting a blood vessel signal with a sensor. A physiologic time interval can be determined, and information related to the physiologic status of the subject can be analyzed and communicated. Pulse wave velocity and time intervals can be calculated using points within a single cardiac cycle and/or using a single cardiac signal from a single detection location.

In other embodiments, a device for monitoring a physiologic status of a human or animal subject is provided. The device includes a blood vessel signal detection module, a processing module in communication with the blood vessel signal detection module configured to determine a physiologic time interval from the blood vessel signal, and a communication module in communication with the processing module configured to communicate information related to the physiologic status of the subject based on the time interval.

In still further embodiments, methods according to the invention include (a) positioning a device having an array of sensing elements on a human or animal subject; (b) detecting a blood vessel signal with the array of sensing elements; (c) comparing the detected signal to predetermined selection criterion; (d) selecting the signal satisfying the criterion; and (e) repeating steps (b)-(d) and storing at least one amplitude and time value for each selected signal.

In certain embodiments, the device includes a plurality of closely spaced sensing elements forming an array on a flexible substrate that detects pulse wave signals from a subject's cardiovascular system. Each sensing element is movable relative to other sensing elements in the array so that the array is conformable to an irregular surface. A signal processor is in communication with the sensing elements and is configured to calculate a pulse travel time based on the pulse wave signals. Embodiments of the invention can provide an inexpensive, automatic device for non-invasively detecting acute physiologic changes associated with blood pressure that could be a life-saving device beneficial to emergency medicine. Embodiments of the invention can also provide a useful tracking tool for diagnostic and other patient- or exercise-monitoring applications.

In other embodiments according to the invention, a method for monitoring pulse wave signals includes positioning a device having a plurality of closely spaced sensing elements forming an array on a flexible substrate on a subject. A pulse wave signal from the subject's cardiovascular system is detected from the sensing elements. A first pulse wave time and a second pulse wave time can be determined. One of the first and second pulse wave times is determined from the pulse wave signal. A pulse wave transit interval from the first and second pulse wave times is then determined.

### BRIEF DESCRIPTION OF THE FIGS

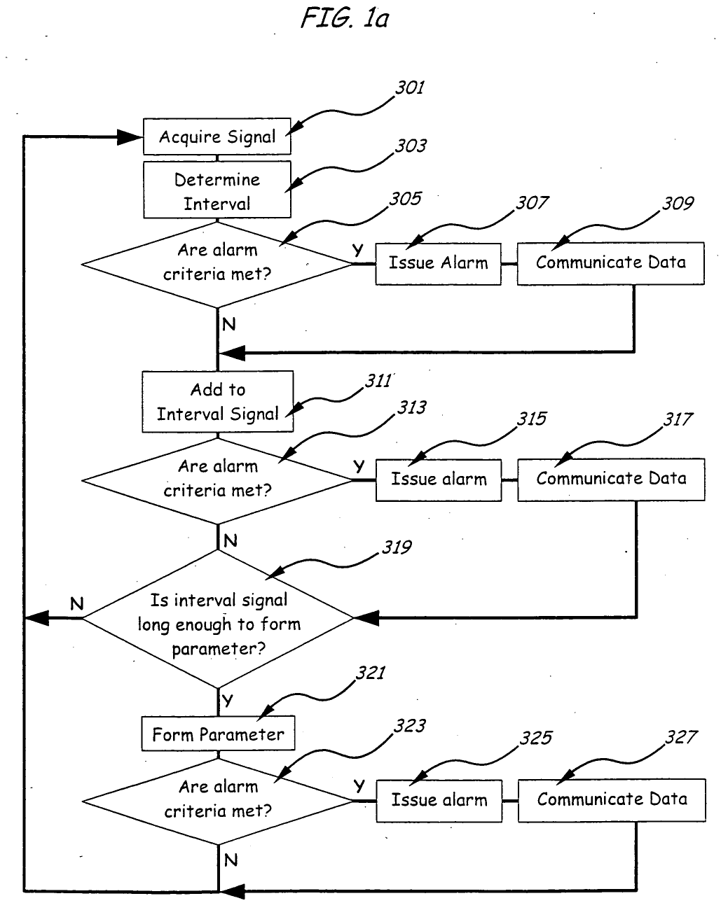

FIG. 1a is a block diagram of the steps of an embodiment of the method of the invention.

FIG. 1b is a block diagram of the steps of another embodiment of the method of the invention.

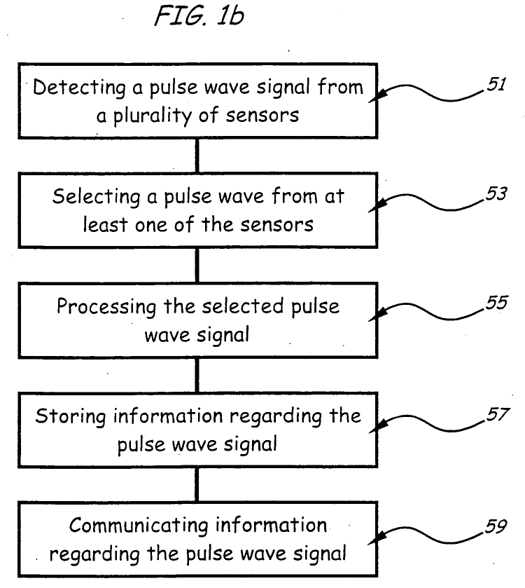

FIG. 2 is a graph of blood velocity and an EKG signal as a function of time the can be used to determine intervals according to embodiments of the invention.

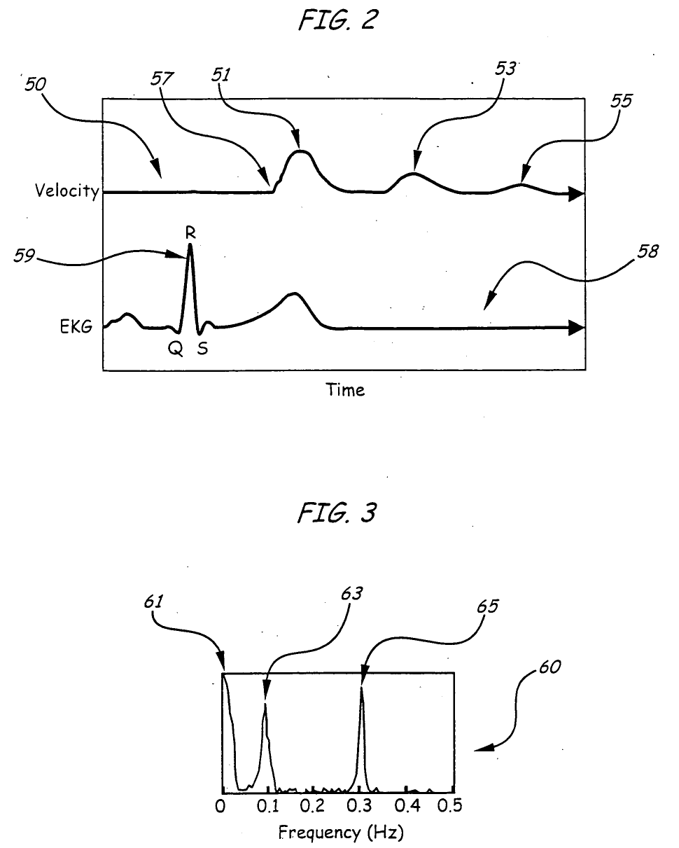

FIG. 3 is a power spectrum according to embodiments of the invention having DC, low frequency and high frequency bands.

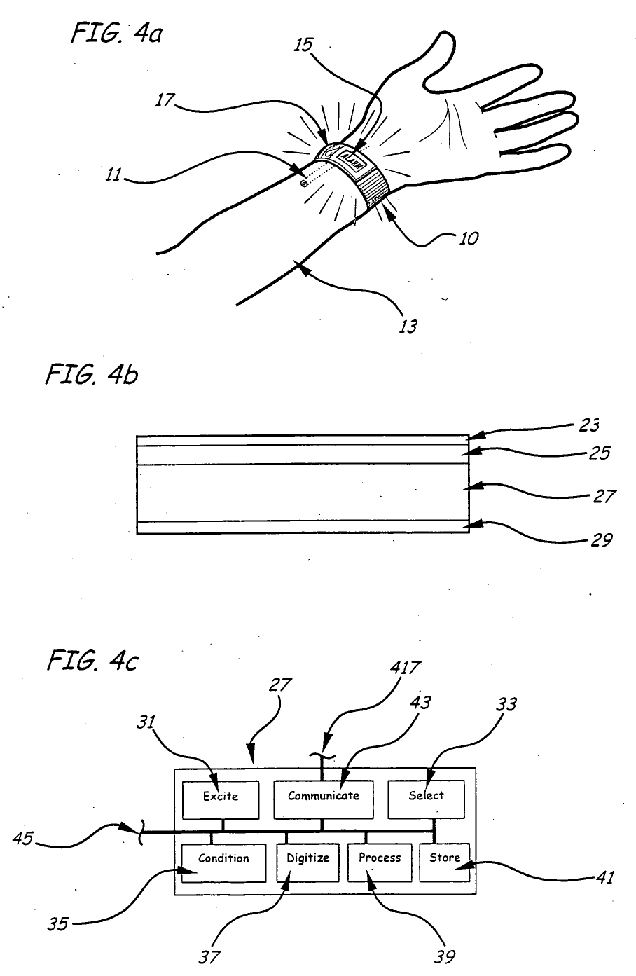

FIG. 4a is a schematic illustration showing a device according to embodiments of the invention in position on a subject's wrist.

FIG. 4b is a schematic illustration of a device according to embodiments of the invention comprising couplant, array, electronics and display components.

FIG. 4c is a block diagram illustrating electronics modules according to embodiments of the invention.

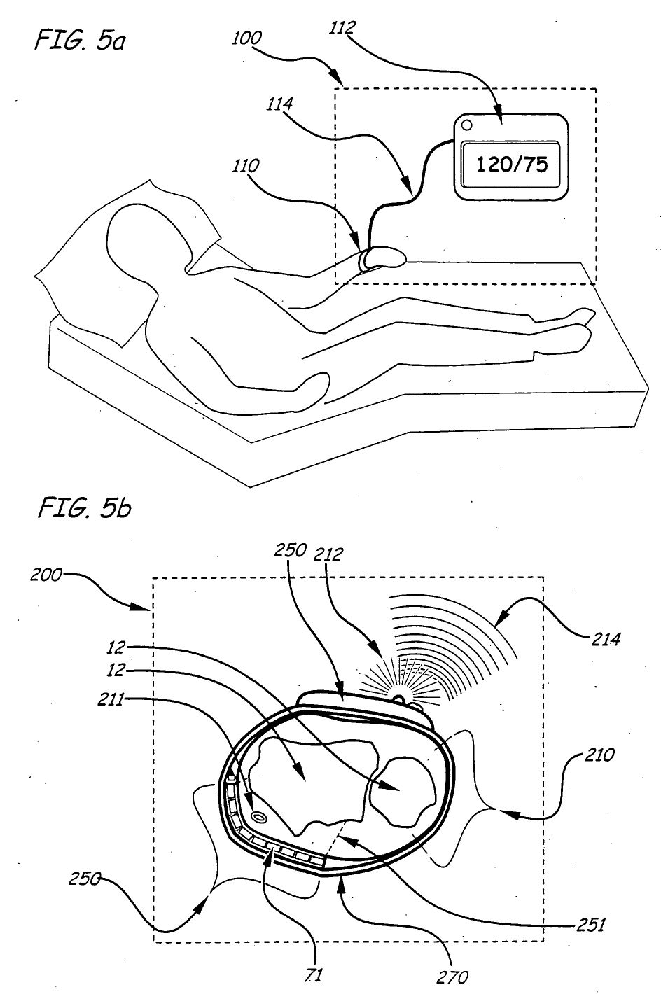

FIG. 5a is a schematic illustration showing alternative embodiments according to the invention in position on a subject.

FIG. 5b is a cross sectional view of a device on a subject's wrist according to embodiments of the invention.

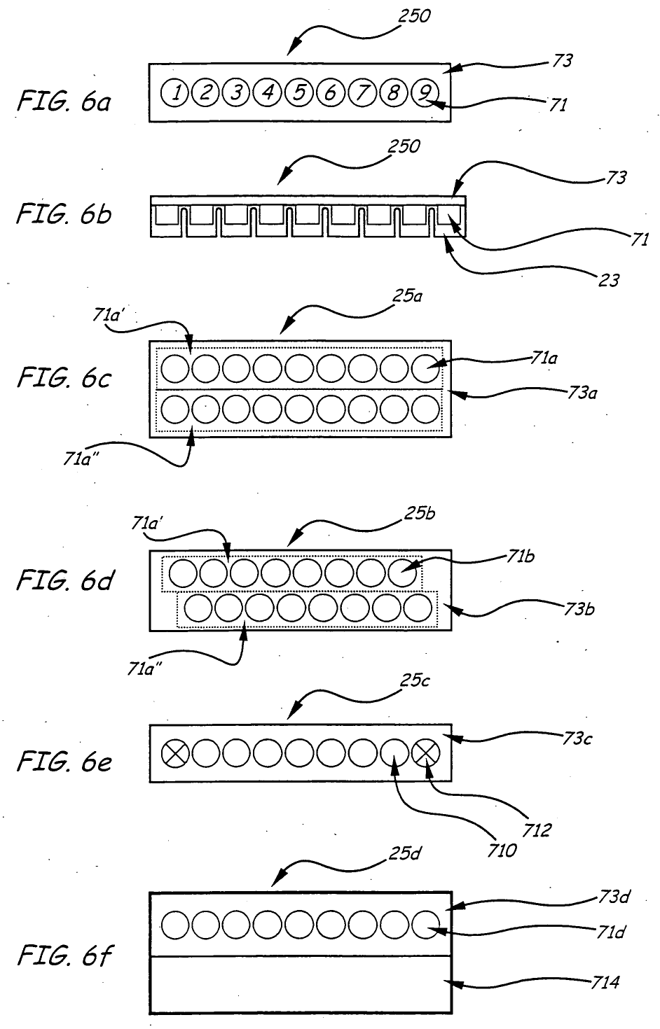

FIG. 6a is a schematic front view of a linear array of sensing elements on a substrate according to embodiments of the invention.

FIG. 6b is a schematic cross sectional view of an array of sensing elements attached to a substrate and covered with a couplant according to embodiments of the invention.

FIG. 6c is a schematic front view of a multi-linear arrangement of sensing elements according to embodiments of the invention.

FIG. 6d is a schematic front view of a staggered arrangement of sensing elements according to embodiments of the invention.

FIG. 6e is a schematic front view of an array having two types of sensing elements according to embodiments of the invention.

FIG. 6f is a schematic front view of an array having a mixed arrangement of sensors according to embodiments of the invention.

### DETAILED DESCRIPTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, layers, components, or features may be exaggerated for clarity.

In certain embodiments, the invention is directed to a noninvasive device and method for continuously monitoring for onset of hypotensive shock or other changes in patient condition, including blood pressure and cardiovascular stability. In some embodiments, the invention comprises a motion-tolerant, automated, non-invasive device, and methods of use, for continuously monitoring cardiovascular signals for real time detection of changes in blood pressure, such as development of hypotensive shock, and other adverse physiologic changes, and for issuance of alarms to enable medical response. Certain embodiments according to the invention are directed to issuing alarms based on time intervals and parameters derived from cardiovascular signals. Other embodiments according to the invention involve using a motion tolerant noninvasive device to monitor the onset of hypotensive shock and of other changes in patient condition, such as blood pressure and cardiovascular stability and to communicate alarms and information related to such changes. Devices according to embodiments of the invention can include sensing arrays that can detect pulse wave arrival time by a various methods, including Doppler velocity sensing, and process the detected signal to extract a first time to be processed together with a second time to calculate a pulse travel time or interval indicative of blood pressure or other measure of cardiovascular status. Other methods according to embodiments of the invention include processing a signal such as a velocity signal to detect acute changes, including cardiac output and changes in cardiac output. For example, the blood velocity signal can be integrated over each of a plurality of heartbeat cycles and assess the magnitude, variability, and aspects of change, e.g. magnitude and rate, in cardiac output. Embodiments of the invention can be useful in treating various conditions, including various cardiac conditions, trauma, autonomic neuropathy, and cardiac tamponade.

In some embodiments, pulse travel time and/or blood velocity time intervals can be determined using a single sensor, a single sensing location, and/or a single cardiovascular signal. The need for multiple signal sensing locations and/or signals may be eliminated. In certain embodiments, pulse travel time and/or blood velocity time intervals can be determined from the signal detected during a single cardiac cycle.

Embodiments of the invention can include a flexible, compliant-faced array of ultrasonic sensing elements that can prevent the signal disruption due to motion artifacts including significant patient movement. The array can detect pulse wave arrival time and process the signal with a second time signal to calculate pulse travel time, which is indicative of systolic blood pressure. Signals from the sensing elements can be processed in other ways to detect acute adverse changes.

For ease of discussion, embodiments of the present invention may be described in specific terms, for example as an ultrasound device, but should not be considered restricted to specific embodiments disclosed herein. For example, various types of sensors, such as optical, mechanical, acoustic, electromechanical, impedance, and electrical sensors may also be used.

As will be appreciated by one of skill in the art, the present invention may be embodied as a device, method, data processing system, or computer program product. Accordingly, the present invention or portions thereof may take the form of an entirely hardware embodiment, an entirely software embodiment or an embodiment combining software and hardware aspects, all generally referred to herein interchangeably as a “circuit” or “module.” Furthermore, the present invention may take the form of a computer program product on a computer-usable storage medium having computer-usable program code means embodied in the medium. Any suitable computer readable medium may be utilized including hard disks, CD-ROMs, optical storage devices, a wired or wireless transmission media such as those supporting the Internet or an intranet, or magnetic storage devices.

The present invention is described below with reference to certain flowchart illustrations and/or block and/or flow diagrams of methods, device (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified in the flowchart and/or block and/or flow diagram block or blocks.

Referring to FIG. 1a, a blood vessel signal is acquired from a sensor placed on a subject (Block 301). The signal can be acquired using various types of sensors, including sensors known to those of skill in the art such as ultrasound, optical, electromagnetic, acoustic, mechanical, electrical, and impedance sensors. The sensors can include arrays of sensing elements. The sensor can sense cardiovascular signals, including signals that indicate blood flow, blood velocity, blood pressure, artery pulsation, cardiac electrical activity, cardiovascular sound, displacement, velocity, acceleration, pressure, light voltage, magnetic resonance, and impedance. Signals can be acquired from a sensor placed at one anatomical location, and the sensor can include one or more types of sensing elements to detect one or a combination of cardiovascular signals. A single cardiovascular signal can be used. However, in some embodiments, more than one sensor placed at different anatomical locations can be used. The sensor(s) can monitor cardiovascular signals continuously to detect acute changes in cardiovascular status.

A time interval is determined from the signal (Block 303). The interval can be a pulse transit time (PTT), such as an interval that may be determined as a time between two points in the blood vessel signal. The selection of points to determine a PTT is discussed in greater detail herein with respect to FIG. 2. Next, the time interval is compared to predetermined alarm criteria (Block 305). The alarm criteria can be preset alarm criteria or based on statistically determined parameters. If the alarm criteria are met, an alarm is issued (Block 307). Data about the time interval can also be communicated (Block 309).

A plurality of interval times can be used to form an interval signal, such as a PTT interval time series signal (PTTI), which can be processed to form a PTT- or PTTI-derived parameter. The PTT- or PTTI-derived parameters can also be used to form one or more interval signals. Thus, as shown in FIG. 1a, the interval measurement is added to the time series comprising an interval signal (Block 311). The interval signal can also be analyzed to determine if an alarm should be issued. Additional alarm criteria can be used to determine if the interval signal meets the alarm criteria (Block 313). If the criteria are met, an alarm is issued (Block 315). Additional data can then be communicated (Block 317).

If sufficient data has been collected in the interval signal (Block 319), a parameter can be formed (Block 321). Preferably, the interval signal includes at least four interval times in order to provide sufficient data to form a parameter; however, fewer data points may be sufficient. A parameter can be a statistically determined interval, interval signal, or value derived from an interval or interval signal. Various statistical methods, including statistical process control (SPC) and other methods known to those of skill in the art, can be used to determine the parameter. Examples of suitable statistical methods include the computation of a mean and standard deviation of a PTTI prior to a medical procedure as the basis for tracking patient status or condition using methods of statistical process control such as described in Understanding, Statistical Process Control, D. J. Wheeler, D. S. Chambers, and W. E. Demming (SPC Press, 2^{nd} Ed., 1992). Other statistical methods can include forming a slope using such statistical methods as a least-squares estimator function. The parameter can be used to define alarm criteria. For example, an alarm may be issued if a particular signal is outside of the standard deviation for previous signals. Examples of alarm criteria include computations of mean, variance, standard deviation, standard error of mean, skew, and kurtosis for the interval signal.

If the alarm criteria are met (Block 323), then an alarm is issued (Block 325). Data about the parameters can also be communicated (Block 327), for example, to a user interface or to additional medical devices. In some embodiments, information communicated to a second device can be used to control the device. Examples of devices that can be controlled using such information include infusion devices, dialysis devices, stress test devices, blood pressure measuring devices, and telecommunications devices.

The blood vessel or cardiac signal can be acquired continuously (Block 301) to determine intervals (Block 303), interval signals (Block 311), and parameters (Block 321) as described above. Sensors for acquiring the signal can include sensors placed on the wrist to detect arterial signals, e.g. from the radial artery. Arm, leg, neck, head, foot, hand and joints are also acceptable locations. A blood velocity signal is preferably used, although such signals as pressure, pulsation, vibration, optical absorbance or reflection, impedance, sound or voltage can be used. In the preferred method, a signal is processed to determine at least two times, here termed a first time and a second time, representing cardiovascular events separated in time within a single cardiac cycle.

In certain embodiments, an array of sensors can be placed at a location on a subject and used to detect a pulse wave signal. As shown in FIG. 1b, methods according to embodiments of the invention comprise steps of: detecting a pulse wave signal from a plurality of sensors (Block 51), selecting a pulse wave signal from at least one of the sensors (Block 53), processing the selected pulse wave signal (Block 55), storing the selected pulse wave signal (Block 57), and communicating information regarding the selected pulse wave signal (Block 59).

Detecting (Block 51) preferably comprises energizing sensors in the array and acquiring a signal by pulsed Doppler ultrasound methods that provide demodulated signals. One or more of the detected signals can be selected for recording or processing. Other methods of detecting, such as optical reflection, absorbance, or Doppler methods, as well as acoustic, electrical, mechanical, or magnetic methods, may also be employed. A single signal can be processed or a plurality of signals may be selected and combined by analog or digital methods, e.g., by array processing methods, to produce an enhanced selected signal as a selected signal for subjecting to sampling.

In certain embodiments, selecting (Block 53) includes comparing the detected signals from each of the sensing elements to predetermined selection criterion and selecting the signal from at least one of the sensing elements satisfying the criterion. The detecting step (Block 51) and the selecting step (Block 53) can be repeated and each selected signal can be stored in an array. The resulting array can by a two-dimensional array having at least one amplitude and time value for each selected signal. The selected signals in the array can be selected from different sensors in the array over time and sampled to form a single digital signal. For example, different sensors in the array may detect the optimal signal at different points in time if the sensor array moves relative to the subject. By repeating the detecting step (Block 51) and the selecting step (Block 53), the optimal signal can be compiled and stored regardless of which sensor detects the optimal signal.

In a preferred embodiment selecting (Block 53) includes interrogating array elements in a sequence, beginning with the one providing the previously sampled signal. Interrogating then proceeds in sequence alternatively to either side of that element. For example, if element 5 in a 9 element array was sampled during the prior sampling cycle, interrogating proceeds in the following sequence: 5,4,6,3,7,2,8,1,9. Once the elements have been interrogated, the signal with the highest amplitude signal is selected. Alternatively, the first signal to meet a selection criterion is sampled without energizing additional elements. Example criteria include an adequate signal amplitude or power, or an adequate signal to noise ratio. Interrogating continues until the selection criterion is met, or a malfunction criterion is met, and an alarm is issued. An example of a malfunction criterion can be a condition when no signal is detected within a period of time such as about one second.

In other embodiments, sequential interrogating begins with the last element to produce a selected signal and proceeds in sequence towards the nearest end of the array. For example, beginning with element 7 in a 9-element array, interrogating would occur in the following sequence: 7,8,9,1,2,3,4,5,6. In further embodiments, interrogating proceeds in order of element identification number, e.g., 1,2,3,4,5,6,7,8,9. In still further embodiments, a signal meeting a selection criterion is sampled repeatedly, without interrogation of other array elements.

In certain embodiments, the selected signal is conditioned and sampled by digitizing with between 4- and 32-bit precision to produce a digital sample, comprising a sample value, sampling time and source identifier, e.g., array element number. The steps from detecting through sampling herein are termed a sampling sequence cycle. The sampling sequence cycle is repeated at a rate between 10 Hz and 1 MHz. A plurality of digital samples is used to form a digital signal, which in some embodiments represent a blood velocity profile. The selected signal can be digitally sampled.

Processing the selected pulse wave signal (Block 55) can include determining a time interval between points in the pulse wave signal such as a pulse travel time. Storing the selected pulse wave signal (Block 57) can include storing a series of pulse wave signals and/or time intervals. Such a series is referred to herein as an “interval signal.” The interval signal can include time intervals for a plurality of heartbeat cycles. The heartbeat cycles can be sequential or selected in various orders including even (non-sequential), uneven (non-sequential), and combinations thereof.

Communicating information (Block 59) can include communicating information with a display and/or by issuing an alarm. Methods according to embodiments of the invention can include monitoring cardiovascular signal intervals, such as pulse transit time, to detect and issue alarms regarding onset of hypotensive shock, to detect other acute physiologic changes, such as during drug infusion, or to control devices that can include infusion therapy devices, dialysis devices, stress test devices, as well as radio and telecommunications devices related to issuing alarms. Other devices that can be controlled include a blood pressure measuring device, such as an invasive catheter, oscillometer, tonometer, or other pressure measureing device, that can provide a blood pressure reading for calibration steps of the method. Such devices can be provided as an integrated portion of a device for detecting a pulse wave signal. In some embodiments, such devices are physically separate from a pulse wave signal detection device and may be in communication with the pulse wave signal detection device, for example, via wired or wireless communications connections. Methods may also comprise forming a continuous high resolution record of an interval signal or of systolic or diastolic blood pressure for uses that can include analyzing overnight hypertension, and detecting acute or short-lived physiologic changes in sleeping, ambulatory or exercising individuals. FIG. 2 depicts a blood velocity signal 50 acquired by Doppler ultrasound and an EKG signal 58 for one cardiac cycle interval. The blood velocity signal 50 can be used alone or together with the EKG signal 58 to determine a time interval or pulse wave velocity. The velocity signal 50 includes numerous fiducial points, such as a wave initiation point 57, a first velocity peak 51 (that can be indicative of a pulse wave arrival), a second velocity peak 53 (that can indicate wave reflections), and a third velocity peak 55 (that can indicate additional wave reflections). These and other features of the velocity signal 50 are referred to herein as fiducial points and can reflect various cardiovascular phenomena. Other examples of fiducial points include peak value, minimum value, intermediate value, mean value, average value, zero crossing, predetermined slope, zero slope, and change in slope.

The PTT can be determined using the signal from a single cardiac cycle without requiring the EKG signal 58. For example, the first velocity peak 51 can be designated as a first time and the secondary velocity peak 53 can be designated as a second time. The PTT is the time difference between the first velocity peak 51 and the secondary velocity peak 53. Optionally, other velocity peaks or other fiducial points such as the third velocity peak 55 or the wave initiation point 57 can be used to calculate PTT.

In addition, an R-wave peak 59 of the EKG signal 58 can be used as either a first time or second time in conjunction with a time from another signal (such as fiducial points in the blood velocity signal 50). Alternatively, an optical detector such as a pulse oximeter, or a pulse detector such as a pressure, vibration, or displacement sensor, or an impedance sensor, electrical or electromagnetic flow sensor can be used to acquire signals from which a first time and/or a second time can be determined.

When more than one sensor is employed in the inventive method, one sensor can provide a first time and a second sensor can provide a second time for determining PTT. Typically, sensors can acquire signals representative of cardiovascular phenomena at different anatomical locations, such as the above example with an EKG from the chest and blood velocity at the wrist, using different types of sensor. Alternatively, two sensors of the same type, e.g. pulse detectors, can be placed at different anatomical locations, such as along the radial artery, and the different times at which they detect a pulse can be used as a first time and a second time to determine PTT.

Changes in PTT, PTTI and derived parameters can be used to detect or forewarn of adverse changes in subject health status, including hypotensive shock, myocardial infarct, hypervolemia, hypovolemia, neurological or autonomic instability or disfunction, acute hypertension, intradialytic hypotension, adverse reaction to medical procedures, as well for controlling procedures or devices, such as an infusion pump, or a blood pressure measuring device. Ambulatory or inpatient use may also be provided, including monitoring related to pre- or post-procedure monitoring, maternal or fetal pre-paripartum monitoring, overnight hypertension diagnosis and tracking, and physiologic response to stress testing or exercise. Another alternative uses two types of sensing elements in the same sensor, e.g., Doppler ultrasound sensing elements to sense blood velocity and acoustic sensing elements to sense heart sounds.

In some embodiments, signals may be integrated over the duration of an interval to provide additional measures of medical status. One such interval has the duration of a cardiac cycle, with integration of blood velocity over the interval providing a measure of cardiac output. PTTI for the integrated blood velocity and parameters derived therefrom (such as mean and standard deviation) are stored for subsequent processing and for comparison to alarm criteria.

FIG. 3 depicts a power spectrum 60 for a PTTI, which can be used to form a power ratio (PR) as an indicator of cardiovascular health and homeostasis. One method of determining PR comprises forming a power spectrum 60 of PTTI, filtering out direct current (DC) components 61 and extracting a low frequency power band 63 and high frequency power band 65, the ratio of which represents PR. Alternatively wavelet methods may be used to calculate PR. A plurality of PR values can be used to generate a PR-PTTI, or “PRI”, which may be stored in memory for subsequent processing and communicating. The PR, PRI and derived parameters may be compared to alarm criteria as the basis for issuing alarms.

Sensors other than for Doppler velocity and EKG may be used according to embodiments of the invention, including ultrasound back scatter, mechanical, optical, electromechanical, electro-magnetic, impedance, and acoustic sensors. In an embodiment using an acoustic sensor to detect heart sounds, the sensor can produce a plurality of signals, which can be subjected to coherent signal combining or array processing methods to enhance the signal prior to determining the time of the heart sound. The heart sound time can then be used to determine a first time or a second time for purposes of determining an interval. Array processing methods, including sensor fusion methods, can also be applied to any plurality of signals from any sensing element type or plurality of sensing elements types.

In some embodiments, PTT is converted to a blood pressure signal, preferably systolic, diastolic, mean, or pulse pressure, to facilitate review by medical personnel and others familiar with absolute measures of blood pressure known to those of skill in the art. Conversion can be performed with a conversion function and a calibration value. A calibration value preferably is input to replace a default value, using any appropriate means including invasive catheter, cuff device, oscillometer, tonometer, or other means of quantifying subject blood pressure, including estimation based on patient history. Estimation is particularly acceptable in such applications as physical exercise when no adverse change in condition is anticipated. Such calibration values may be used as alarm criteria. Mean, variance, standard deviation, standard error of mean, skew, and kurtosis for the interval signal can also be computed and subsequently used as alarm criteria. Calibration values may be input by wireless or wire connection, or by manual input.

When a plurality of calibration values is input, an updated conversion function is calculated from time to time and stored for use as an updated conversion function in conjunction with the calibration value in calculating measures of blood pressure. In one embodiment, the two most recent calibration values are used to determine a linear updated conversion function. In an alternative embodiment more than two calibrating values are used to determine an updated conversion function, e.g., by least squares regression analysis. Changes in conversion function parameters are used to form a conversion function signal (CFI), which is stored in memory for further processing and communicating.

The PTT, PTTI, derived parameters, and derived parameter signals (e.g., CFI and PRI), may be compared to alarm criteria and used as the basis for issuing alarms. One such criterion is a PTT indicative of a systolic blood pressure between about 60 and about 120 mm Hg, preferably less than or equal to about 90 mm Hg. Additional criteria include rate, magnitude, and variability of change. Such criteria may also be applied to pulse intervals or EKG R-wave intervals.

Alarm criteria for PTT, PTTI, and other interval signals, derived parameters, and derived parameter signals can be used as the basis for communicating to an external device such as a user interface or medical equipment. The communications to an external device can include the output from comparing data to alarm criteria and/or statistical process control parameters. In some embodiments using statistic process alarming, a portion of PTTI, e.g., representative of patient condition or status, before a procedure, is acquired and processed to determine mean value and range, or other measure of variability such as standard deviation. These calculated values (e.g., mean and range) then become alarm criteria for subsequent portions of PTTI or a subsequent PTTI. When an alarm criterion is met, the alarm condition and other information, including intervals, interval signals, and parameters, can be communicated to medical personnel or others. Communication can include issuing an sound, light or vibration alarm or by sending and alarm signal and other data by wire, fiber-optic, or wireless devices to a user interface.

FIG. 4a depicts a device 10 for noninvasive monitoring of cardiovascular signals, e.g. from an artery 11, at an anatomical location of a subject 13 where a cardiovascular signal is detectable. The device can monitor other locations on the subject 13 such as the neck or ankle. The device 10 includes a housing 15 and an attachment strap 17. The strap 17 optionally can include a positioning segment such as an opening, depression, or rigid segment to accommodate a bony prominence, e.g. the radial or ulnar head in the wrist, to localize and stabilize the device 10.

FIG. 4b is a schematic illustration of the system held by the housing 15, comprising a couplant layer 23, an array 25, an electronics assembly 27, and a display module. The couplant layer 23 can be permanently affixed or detachably affixed to the array 25.

FIG. 4c is a block diagram of the electronics assembly 27, including an excitation module 31 to excite the array using power (for example, battery power), a selection module 33 for selecting signals, a conditioning module 35 to condition a signal, a digitizing module 37, a processing module 39, a storage module 41, a communication module 43 (which can include a module for issuing alarms), an input connector 45 and an output connector 47. The communication module 43 can also include circuits for wireless communication. The display module 29 (FIG. 4b) can comprise an alphanumeric display, an alarm light, an alarm buzzer, and/or buttons for manual data input and control that can display information transmitted from the communication module 43. In some embodiments, the display module is omitted.

FIG. 5a depicts an alternative embodiment 100 of a device according to the invention that can be used in hospitals, clinics or other locations, comprising a band 110 applied to the wrist and connected to a base station 112 by a cable 114 that can conduct power and signals between the band 110 and the base station 112. The base station 112 can include the electronics assembly 27 (FIG. 4c) while the band 110 can include a strap and a sensing array. Alternatively, electronic modules similar to the electronics modules shown in FIG. 4a may be allocated between the band 110 and the base station 112 in various ways. For example, the band 110 can include a sensing array and selection, conditioning, and digitizing modules, while the base station 112 can include an excitation module, processing module, storing module, and communication module. Other allocations of components between the band 110 and the base unit 112 are also acceptable.

FIG. 5b depicts an alternative wireless embodiment of a device 200, comprising a band 210 including an attachment strap 270, an array 250 positioned over the artery 210, and a housing 350 contra lateral to the array 250 to enable wearing with the housing 250, for example on the dorsal surface of the wrist. The strap 270 can conduct power and signals between the array 250 and the housing 250. The strap 270 can also include a positioning segment 210, which can be a relatively rigid segment that attaches to a natural protrusion of tile wrist for localizing and positioning the array 250. The housing 250 can additionally comprise an optical alarm and a speaker 214 to sound an audible alarm. The array 250 can be seen to conform to the surface of the wrist, which is irregular due by the presence of bones 12, and maintain contact and direct the signal over a wide sensing window 251 containing the artery 210. This can increase the motion tolerance of the device 200.

FIG. 6a depicts the face of a preferred embodiment of the array 250. The array 250 can comprise between 1 and about 257 or more sensing elements 71 attached to a substrate 73. Other numbers of sensing elements 71, including 64, 128, and 256 or more than 257 elements, can be used. The sensing elements 71 are preferably ultrasonic transceivers with a fundamental resonance frequency between 10 kHz and 20 Mhz. The substrate 73 preferably is conformable to an irregular surface such that each element 71 of the array 250 is movable with respect to the other sensing elements 71. FIG. 6b depicts a section view of a preferred embodiment of an array, showing sensing elements 71 attached to the substrate 73 and covered with a compliant, signal-permissive couplant layer 75 to be placed in contact with a human or animal subject. The couplant layer 75 can provide additional flexibility to conform the array 250 to an irregular surface.

The preferred type of ultrasonic sensing elements 71 typically is piezoelectric ceramic, although alternative types such as polyvinylidene fluoride (PVDF) film and crystalline solids can be used. Alternatively, any sensing technology can be used that can non-invasively or invasively detect cardiovascular phenomena. Useful types also include acoustic, electrical, electromagnetic, pressure, impedance, and optical.

Sensing elements may be arranged in various fashions other than the linear arrangement depicted in FIG. 6a. FIG. 6c depicts an alternative arrangement as a multi-linear array 25a of sensing elements 71a on a substrate 73a. FIG. 4d depicts another array 25b having the sensing elements 71b are arranged as a staggered array 26b on substrate 73b. The array 25b preferably has a λ/4 offset, where λ is the wavelength of the interrogation pulse of an ultrasound sensor.

The sensing elements operate preferably in pulsed mode, although continuous mode is also acceptable. For continuous mode, sensing elements such as sensing elements 71a and 71b configured as shown in FIGS. 6c and 6d, can be divided into two groups: a transmitting group 71a′, 71a″ and a receiving group 71a″, 71b″ for transmitting and receiving ultrasound signals. Pulsed ultrasound can be used to measure blood velocity by Doppler methods or to measure artery pulsation by back scatter methods or by Doppler imaging. Continuous ultrasound employs Doppler methods to measure either blood velocity or artery pulsation.

The array may alternately comprise of more than one type of sensing element as shown in FIG. 6e. One example is an array 25c comprising a substrate 73c to which are attached optical sensing elements 710 that can detect hemoglobin content in an artery, and acoustic sensing elements 712 to detect heart sounds. FIG. 6f depicts another exemplary embodiment comprising ultrasonic sensing elements 71d on substrate 73d. The ultrasound sensing elements 71d are formed of a piezoelectric ceramic and a film sensing element 714 composed of a piezoelectric film Such as PVDF. The film sensing element 714 can be used to emit signals with the sensing elements 71d detecting return signals. Alternatively, the function of the sensor types can be reversed.

The foregoing embodiments are illustrative of the present invention, and are not to be construed as limiting thereof. The invention is defined by the following claims, with equivalents of the claims to be included therein.

1. A method of monitoring a physiologic status of a human or animal subject, comprising:
(a) detecting a blood vessel signal with a sensor;
(b) determining a physiologic time interval from the signal; and
(c) communicating information related to the physiologic status of the subject based on the time interval.

2. The method of claim 1, further comprising:
(d) comparing the time interval to predetermined alarm criteria; and
(e) issuing an alarm in step (c) if the alarm criteria are met, wherein the information in step (c) comprises the alarm.

3. The method of claim 1, wherein step (b) further comprises determining the difference between a first time and a second time of two fiducial points related to a single heartbeat cycle.

4. The method of claim 1, further comprising:
(d) repeating steps (a), (b), and (c) and thereby collecting a series of time intervals;
(e) determining an interval signal, wherein the interval signal comprises a portion of the series of time intervals; and
(f) deriving parameters from the interval signal.

5. The method of claim 4, further comprising:
(g) comparing at least one of: a time interval, interval signal, and derived parameter; and
(h) forming alarm criteria from the comparing step.

6. The method of claim 3, wherein the first time and second time are fiducial time points in pulse propagation.

7. The method of claim 6, wherein the fiducial time point is a time point indicative of at least one of: pulse wave initiation, pulse wave arrival and pulse wave reflection.

8. The method of claim 1, wherein the sensor has at least two sensing elements.

9. The method of claim 8, wherein the sensing elements employ different sensing element types.

10. The method of claim 1, further comprising
(d) repeating steps (a), (b), and (c) and thereby collecting a series of time intervals;
(e) determining an interval signal, wherein the interval signal comprises a portion of the series of time intervals;
(f) deriving parameters from the interval signal;
(g) computing at least one of: mean, variance, standard deviation, and standard error of mean for the interval signal; and
(h) determining alarm criteria from the at least one of mean, variance, standard deviation, standard error of mean, skew, and kurtosis.

11. The method of claim 1, wherein the sensor is noninvasive.

12. A device for monitoring a physiologic status of a human or animal subject comprising:
a blood vessel signal detection module that detects a blood vessel signal;
a processing module in communication with the blood vessel signal detection module configured to determine a physiologic time interval from the blood vessel signal; and
a communication module in communication with the processing module configured to communicate information related to the physiologic status of the subject based on the time interval.

13. The device of claim 12, wherein the processing module is further configured to select a first time and a second time during a single heartbeat cycle, wherein the time interval is determined by the difference between a first time and a second time.

14. The device of claim 12, wherein the detection module is in communication with a sensor, wherein the sensor comprises at least one of: ultrasonic, acoustic, electric, impedance, electromagnetic, optical, electromechanical, and mechanical sensors.

15. The device of claim 12, where the detection module is configured to detect at least one of: displacement, velocity, acceleration, voltage, pressure, and sound for at least one of: blood, artery, and heart.

16. The device of claim 12, wherein the sensor comprises an array of sensing elements, and further comprising a selection module configured to select one or more signals from the array.

17. The device of claim 16, wherein the sensing elements in the array comprise more than one type of sensing element.

18. The device of claim 16, wherein the array is conformable.

19. The device of claim 16, wherein the array comprises an emitting face, and further comprising a conformable, signal-transmissive couplant layer on the emitting face of the array.

20. The device of claim 19, wherein the couplant layer is detachably attached to the emitting face of the array.

21. The device of claim 19, wherein the couplant layer is attached to the emitting face of the array.

22. A method for selecting among a plurality of cardiovascular signal comprising:
(a) positioning a device having an array of sensing elements on a human or animal subject;
(b) detecting blood vessel signals with the array of sensing elements;
(c) comparing the detected signals to predetermined selection criterion;
(d) selecting a signal from the detected signals that satisfies the criterion; and
(e) repeating steps (b)-(d) and storing at least one amplitude and time value for each selected signal.

23. The method of claim 22, wherein the predetermined selection criterion comprises selecting a sensing element in the array that detects a signal with the highest amplitude compared to the other sensing elements in the array.

24. The method of claim 22, wherein the predetermined selection criterion comprises selecting a sensing element in an array sequence that is the first sensing element in the sequence to detect a signal having an amplitude exceeding a predetermined value.

25. The method of claim 22, wherein the predetermined selection criterion comprises selecting a sensing element in the array that has a signal with the highest signal/noise ratio compared to the other sensing elements in the array.

26. The method of claim 22, wherein the predetermined selection criterion comprises selecting a sensing element in an array sequence that is the first sensing element in the sequence to detect a signal having a signal/noise ratio exceeding a predetermined value.

27. The method of claim 22, wherein step (b) further comprises interrogating each sensing element in the array in a predetermined sequence.

28. The method of claim 27, wherein step (e) further comprises repeating steps (b)-(d) using a plurality of predetermined sequences to select a plurality of signals, wherein each predetermined sequence begins with the sensing element selected from the previous sequence.

29. A noninvasive device for detecting pulse wave signals comprising:
a plurality of closely spaced sensing elements forming an array on a flexible substrate that detect pulse wave signals from a subject's cardiovascular system, wherein each sensing element is movable relative to other sensing elements in the array so that the array is conformable to an irregular surface; and
a signal processor in communication with the sensing elements configured to calculate a pulse travel time based on the pulse wave signals.

30. The device of claim 29, wherein the signal processor is further configured to issue an alarm if the calculated pulse travel time meets predetermined criteria indicating the onset of hypotensive shock.

31. The device of claim 29, further comprising a couplant layer on a side of the sensing elements facing away from the substrate that further conforms the sensing elements to a subject.

32. The device of claim 29, further comprising a signal selection module configured to select a pulse wave signal from one of the sensing elements having the highest amplitude.

33. The device of claim 32, wherein the signal selection module is further configured to interrogate the sensing elements in a series of sequences, wherein each sequence begins with the selected sensing element having the highest amplitude from the previous sequence and proceeds with the other sensing elements in alternative serial order to either side of the selected sensing element, with the sequence proceeding from proximate the selected sensing element to the ends of the array.

34. The device of claim 32, wherein the signal selection module is further configured to interrogate the sensing elements in a series of sequences, wherein each sequence begins with the selected sensing element having the highest amplitude from the previous sequence and proceeds with the other sensing elements in serial order toward the nearest end of the array.

35. The device of claim 32, wherein the signal selection module is further configured to select the first signal to meet a predetermined selection criterion.

36. The device of claim 29, further comprising a band configured to attach the array to an appendage of the subject.

37. A method for monitoring cardiovascular signals comprising:
(a) positioning a device having a having a plurality of closely spaced sensing elements forming an array on a flexible substrate on a subject;
(b) detecting a blood vessel signal from the subject's cardiovascular system with the sensing elements;
(c) determining a first pulse wave time and a second pulse wave time, wherein one of the first and second pulse wave times is determined from the blood vessel signal; and
(d) determining a pulse wave transit interval from the first and second pulse wave times.

38. The method of claim 37, further comprising:
(e) comparing the pulse wave transit interval to predetermined alarm criteria; and
(f) issuing an alarm if the pulse wave transit interval meets the predetermined criteria.

39. The method of claim 37, further comprising:
(e) repeating steps (b)-(d) to determine an interval signal from a plurality of pulse wave transit intervals;
(f) comparing the interval signal to predetermined alarm criteria; and
(g) issuing an alarm if the interval signal meets the predetermined alarm criteria.

40. The method of claim 37, wherein the alarm criteria comprises at least one of interval signal magnitude, variability, and rates of changes thereof.

41. The method of claim 37, wherein the alarm criteria further comprise an interval power ratio used to monitor patient condition and reaction.

42. The method of claim 37, wherein one of the first and second pulse wave times is determined from an EKG signal and the other of the first and second pulse wave times is determined from the blood vessel signal.

43. The method of claim 37, wherein the blood vessel signal comprises a fiducial point from at least one of a pulse wave signal, a blood velocity signal, and an artery wall displacement signal.

44. The method of claim 37, wherein one of the first and second pulse wave times is a pulse wave arrival time and the other of the first and second pulse wave times is a reflected pulse wave arrival time.

45. The method of claim 37, wherein one of the first and second pulse wave times is a pulse wave arrival time and the other of the first and second pulse wave times is a time related to aortic valve action.
